(19) United States
(12) Patent Application Publication
(10) Pub. No.: US 2009/0248150 A1
(43) Pub. Date: Oct. 1, 2009

(54) HYDROGEL INTRAOCULAR LENS AND METHOD OF FORMING SAME

(76) Inventor: Chance Lehman, Dallas, TX (US)

Correspondence Address: ALCON, IP LEGAL, TB4-8, 6201 SOUTH FREEWAY, FORT WORTH, TX, 76134, US

(21) Appl. No.: 12/402,865

(22) Filed: Mar. 12, 2009

Related U.S. Application Data
(60) Provisional application No. 61/039,896, filed on Mar. 27, 2008.

(51) Int. Cl. A61F 2/16

(52) U.S. Cl. 623/6.6; 523/113

(57) ABSTRACT

The present invention is directed ophthalmic device materials and, more particularly to an intraocular lens (IOL) formed of a hydrogel material. The hydrogel material includes an acrylate polymer and a radiation resistant compound. The material typically has a relatively high refractive index and/or exhibits a desired degree of radiation protection.

### CROSS-REFERENCE TO RELATED APPLICATION

The present application claims priority based on U.S. Provisional Patent Application Ser. No. 61/039,896 filed Mar. 27, 2008.

### TECHNICAL FIELD OF THE INVENTION

The present invention is related to ophthalmic device materials and, more particularly to an intraocular lens (IOL) formed of an acrylate hydrogel material having a desired refractive index, a desired degree of radiation protection, desired ion permeability or a combination thereof.

### BACKGROUND OF THE INVENTION

The present invention is directed to ophthalmic devices and particularly intraocular lenses (IOLs). IOLs have been developed and inserted into various locations of the eye and can be used to supplement or correct the vision provided by the natural crystalline lens of the eye or can replace the natural crystalline lens of the eye. Lenses that supplement or correct the vision without replacing the natural crystalline lens are typically referred to as Phakic Lenses while lenses that replace the natural crystalline lens are typically referred to as Aphakic lenses. Phakic lenses can be located within the anterior chamber (AC) of the eye (AC Phakic lenses) or the posterior chamber (PC) of the eye (PC Phakic Lenses).

IOLs can be formed of a variety of materials. Recently, however, there has been a trend toward using soft, foldable materials, which tend to be easier to insert into the eye through a small incision in the eye. Generally, the materials of these lenses fall into the following categories: hydrogels; silicones, and non-hydrogel acrylics.

It is typically desirable for the material of an IOL to have a relatively high refractive index so that the IOL can remain relatively thin and still exhibit a relatively high degree of vision correction. This is particularly the case for PC Phakic lenses. Historically, however, hydrogel materials have typically exhibited undesirably low refractive indexes. Therefore, researchers have invested time and effort toward the discovery of hydrogel materials that have higher refractive indexes. Examples of such materials are discussed in U.S. Pat. Nos. 4,036,814; 4,123,407; 4,123,408; 4,430,458; 4,495,313; 4,680,336; 4,620,954; 4,749,761; 4,866,148; 4,889,664 5,135,965; 5,824,719; 5,936,052; 6,015,842; and 6,140,438 and U.S. Patent Publication; 2002/0128417, all of which are fully incorporated herein by reference for all purposes.

While these new materials have provided desired refractive indexes, they also have drawbacks. In particular, it has been found that these materials can exhibit undesirable degrees of degradation when exposed to electromagnetic radiation and particularly ultraviolet (UV) radiation. Such degradation can inhibit or degrade the ability of an IOL in correcting an individual's vision and can potentially cause other vision issues such as “cloudy” vision or spots.

Many compounds (e.g., UV chromophores) are known and have been incorporated into ophthalmic lenses (e.g., IOLs and contact lenses) for protecting eye tissue from harmful electromagnetic radiation. These compounds can absorb harmful UV radiation such that it does not reach the tissue of the eye. At the same time, however, these compounds typically do not protect the ophthalmic lenses from harmful radiation and can, in many instances, accelerate degradation of the ophthalmic lenses since the harmful rays are absorbed within the lense. This type of degradation can be particularly detrimental for IOLs since such lenses are typically implanted into the eye for extended periods of time and, during those periods of time, radiation can undesirably change characteristics (e.g., refractive index, power, transmission ability or the like) of the lenses.

Hydrogel lenses can be quite susceptible to degradation caused by UV and other radiation. Moreover, such degradation can be increased by the inclusion of certain UV chromophores in the lenses. There exist very few protective compounds that are suitable for use in hydrogel IOLs (particularly P.C. Phakic IOLs) where those compounds do not increase such degradation or where the compounds protect the IOL material from harmful electromagnetic radiation.

Additionally, hydrogel lenses have often required relatively high concentrations of the UV chromophores to assure a desired degree of UV absorption. However, such concentrations can reduce ion permeability or other desired properties of the lens.

Thus, there is a need for a hydrogel IOL that incorporates an effective UV resistant compound where the material of that IOL exhibits a desired degree of resistance to degradation that might otherwise be caused by exposure to electromagnetic radiation. Moreover, it would be particularly desirable for that IOL material to exhibit a relatively high refractive index, relatively high ion permeability and/or a relatively low loss of refractive index and/or power due to radiation exposure.

### SUMMARY OF THE INVENTION

The present invention is directed to hydrogel material suitable for use as an IOL as well as IOLs formed with the material. The intraocular lens can be configured for insertion into the posterior chamber or the anterior chamber of the eye and can be configured as a Phakic or Aphakic lens. Preferably, however, the lens is configured in size and shape as a P.C. Phakic lens. The lens and/or hydrogel material of the lens is typically formed with cross-linked acrylate polymer. The lens and/or hydrogel material also typically includes UV chromophore and the UV chromophore typically includes a benzotriazole (e.g., a 2(-2 hydroxyphenyl)benzotriazole). Preferably, the UV chromophore significantly enhances resistance of the lens to degradation by electromagnetic radiation.

UV chromophores suitable for use in the ophthalmic device materials of the present invention are represented by formula (A).

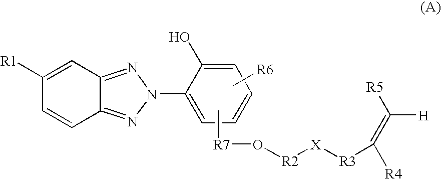

wherein for formula (A)
R₁ is a substituted or unsubstituted C₁-C₆ alkyl, a halogen, OH, C₁-C₁₂ alkyloxy, optionally substituted phenoxy, or optionally substituted napthyloxy, where the optional substituents are C₁-C₆ alkyl, C₁-C₆ alkoxy, OH, —(CH₂CH₂O)ₙ—, or —(CH₂CH(CH₃)O)ₙ—;
R₂ is a C₁-C₁₂ alkyl, (CH₂CH₂O)ₙ, (CH₂CH(CH₃)O)ₙ, or CH₂CH₂CH₂(Si(CH₃)₂O)ₘSi(CH₃)₂CH₂CH₂CH₂;
X is nothing if R₂ is (CH₂CH₂O)ₙ or (CH₂CH(CH₃)O)ₙ, otherwise X is O, NR₄, or S;
R₃ is nothing, C(═O), C(═O)CⱼH₂ⱼ, C₁-C₆ alkyl, phenyl, or C₁-C₆ alkylphenyl;
R₄ is H or methyl;
R₅ is H, C₁-C₆ alkyl, or phenyl;
R₆ is H, C₁-C₁₂ alkyl, or C₁-C₁₂ alkyloxy (e.g., methoxy);
R₇ is C₁-C₆ alkyl or nothing;
m is 1-9;
n is 2-10; and
j is 1-6.

In preferred embodiments of the invention, polymer material includes a first monomer comprised of one or more nitrogen-containing monomers, preferably cyclic and most preferably heterocyclic nitrogen containing monomers. It is contemplated that the polymeric material of the hydrogel can include vinyl methacrylate and more particularly can include an NVP methacrylate copolymer. In a highly preferred embodiment, the vinyl methacrylate includes an NVP-co-hydroxyl methacrylate, an NVP-co-aryl mecthacrylate or a combination thereof.

### DETAILED DESCRIPTION OF THE INVENTION

The present invention is predicated upon the provision of an intraocular lens (IOL) that is formed of hydrogel material and that includes a radiation resistant ingredient or compound (i.e., a UV (ultraviolet) chromophore). The UV resistant compound will typically assist the hydrogel material in resisting degradation that it might otherwise experience due to exposure to electromagnetic radiation, particularly UV radiation.

As used herein, the term “hydrogel” or “hydrogel material” means a material that includes greater than 30% by weight water when that material is located within an aqueous environment in a human eye.

As used herein, the term “electromagnetic radiation” includes all light in the electromagnetic spectrum whether visible or non-visible.

The hydrogel material used to form the IOL of the present invention will typically include polymeric material. The polymeric material can be composed of one polymer or a variety or mixture of polymers. The polymeric material can include thermoplastic polymer and will typically include thermoset or thermosettable polymer. the polymeric material can include polymers of a single repeat unit, copolymers or both.

Preferably, the polymeric material of the hydrogel includes or is formed partially, entirely or substantially entirely of a copolymer component that is comprised of copolymers having a mixture of first monomer and second monomer.

In preferred embodiments of the invention, the first monomer can be comprised of nitrogen-containing monomers, preferably cyclic and most preferably heterocyclic nitrogen containing monomers. Heterocyclic N-vinyl monomers are especially preferred, for example N-vinyl lactams. Preferred N-vinyl lactams are pyrrolidone, piperidone and caprolactam and their derivatives, such as N-vinyl-2-piperidone, N-vinyl-2-pyrrolidone, N-vinyl caprolactam or derivatives thereof. It is contemplated that at least 80%, 90% or more by weight of the first monomer can be composed of any one or any combination of these monomers.

As an addition or alternative to N-vinyl lactams, heterocyclic N-vinyl monomers such as N-vinyl imidazole, N-vinyl succinamide or N-vinyl glutarimide may be employed.

Alternative or additional nitrogen-containing monomers to the heterocyclic monomers referred to above are amido derivatives of (meth) acrylic compounds, for example a (meth) acrylamide or an N-substituted derivative thereof. Preferred are those which are mono- or di-substituted with, for example alkyl, hydroxyalkyl or aminoalkyl substituents. Specific examples of such materials are N-methyl acrylamide, N-isopropyl acrylamide, N-diacetone acrylamide, N,N-dimethyl acrylamide, N,N-dimethylaminomethyl acrylamide, N,N-dimethylaminoethyl acrylamide, N-methylaminoisopropyl acrylamide or a methacrylamide analog of any one of the foregoing.

The second monomer of the copolymer material is typically of formulation 1 below:

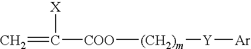

wherein: X is H or CH₃;
m is 0-10;
Y is nothing, O, S, or NR wherein R is H, CH₃, CₙH₂ₙ₊₁(n=1-10), iso-OC₃H₇, C₆H₅, or CH₂C₆H₅;
Ar is any aromatic ring which can be unsubstituted or substituted with CH₃, C₂H₅, n-C₃H₇, iso-C₃H₇, OCH₃, C₆H₁₁, C₆H₅, or CH₂C₆H₅;

Suitable monomers of structure (I) include, but are not limited to: 2-ethylphenoxy methacrylate; 2-ethylphenoxy acrylate; 2-ethylthiophenyl methacrylate; 2-ethylthiophenyl acrylate; 2-ethylaminophenyl methacrylate; 2-ethylaminophenyl acrylate; phenyl methacrylate; phenyl acrylate; benzyl methacrylate; benzyl acrylate; 2-phenylethyl methacrylate; 2-phenylethyl acrylate; 3-phenylpropyl methacrylate; 3-phenylpropyl acrylate; 4-phenylbutyl methacrylate; 4-phenylbutyl acrylate; 4-methylphenyl methacrylate; 4-methylphenyl acrylate; 4-methylbenzyl methacrylate; 4-methylbenzyl acrylate; 2-2-methylphenylethyl methacrylate; 2-2-methylphenylethyl acrylate; 2-3-methylphenylethyl methacrylate; 2-3-methylphenylethyl acrylate; 24-methylphenylethyl methacrylate; 2-4-methylphenylethyl acrylate; 2-(4-propylphenyl)ethyl methacrylate; 2-(4-propylphenyl)ethyl acrylate; 2-(4-(1-methylethyl)phenyl)ethyl methacrylate; 2-(4-(1-methylethyl)phenyl)ethyl acrylate; 2-(4-methoxyphenyl)ethyl methacrylate; 2-(4-methoxyphenyl)ethyl acrylate; 2-(4-cyclohexylphenyl)ethyl methacrylate; 2-(4-cyclohexylphenyl)ethyl acrylate; 2-(2-chlorophenyl)ethyl methacrylate; 2-(2-chlorophenyl)ethyl acrylate; 2-(3-chlorophenyl)ethyl methacrylate; 2-(3-chlorophenyl)ethyl acrylate; 2-(4-chlorophenyl)ethyl methacrylate; 2-(4-chlorophenyl)ethyl acrylate; 2-(4-bromophenyl)ethyl methacrylate; 2-(4-bromophenyl)ethyl acrylate; 2-(3-phenylphenyl)ethyl methacrylate; 2-(3-phenylphenyl)ethyl acrylate; 2-(4-phenylphenyl)ethyl methacrylate; 2-(4-phenylphenyl)ethyl acrylate; 2-(4-benzylphenyl)ethyl methacrylate; and 2-(4-benzylphenyl)ethyl acrylate, and the like.

Preferred monomers of structure (I) are those wherein m is 2-4, Y is nothing or O, and Ar is phenyl. Most preferred are 2-phenylethyl acrylate, 2-phenylethyl methacrylate and combinations thereof. It is contemplated that at least 80%, 90% or more by weight or more of the second monomer is composed of one or both of these two monomers.

It is to be understood that the copolymer component formed of the first monomer and the second monomer can include a variety of different copolymers having any of the monomers mentioned within the group of monomers suitable as the first monomer and any of the monomers mentioned within the group of monomers suitable as the second monomer. The copolymer component can also be formed of singular copolymer. Preferred copolymers suitable for the copolymer component include, without limitation, N-vinyl-2-pyrrolidone-co-yl methacrylate, N-vinyl-2-pyrrolidone-co-hydroxyl(alkyl)methacrylate or combinations thereof.

The copolymer component is typically at least 30%, more typically at least 60% and even more typically at least 80% or even at least 90% by weight of the polymeric material or the hydrogel material that forms the IOL. The copolymer component is also typically less than about 99.5% by weight of the hydrogel material that forms the IOL. Unless otherwise stated, the percentages (e.g., weight percentages) for the ingredients of the hydrogel material are done as anhydrous percentages or percentages that do not include water or other aqueous medium that would typically permeate the hydrogel upon exposure to an aqueous medium environment. Such hydrogel material is typically entirely solid for such weight percentages prior to exposure to such aqueous medium.

A curing agent (e.g., initiator) is typically employed to initiate the polymerization of the monomers and/or carry out the cross-linking or thermosetting of the polymers (e.g., copolymers) formed of those monomers. Examples of suitable curing agents include peroxy curing agents (i.e., any curing agent including a peroxy group), oxide curing agents (i.e., any curing agent include an oxide group (e.g., a dioxide) or others known by the skilled artisan. One example of a preferred peroxy curing agent is a tert-butyl peroxy-2-ethylhexanoate organic peroxide initiator. Such curing agent is particularly suitable for thermal cure. One example of an oxide curing agent is 2,4,6-Trimethylbenzoyldiphenylphosphine oxide. Such curing agent is particularly suitable for blue light cure.

Curing agent accelerators may also be employed. Various curing agents accelerators are known and can be used in prescribed amounts or amounts experimentally found to be suitable. Typically, amounts of the curing agent, the curing agent accelerator or a combination thereof are between about 0.1% and about 8% by weight of the hydrogel material.

Curing agents and accelerators can be used in various amounts, which will typically depend upon the monomers and polymers being employed, any ambient conditions (e.g., heat, light or otherwise) being used for curing and/or other factors.

As discussed above, the hydrogel material of the present invention includes radiation resistant compound. The radiation resistant compound can be a singular compound or can be a mixture of multiple compounds.

As used herein, a “radiation resistant compound” is a compound that assists that hydrogel material, particularly the polymeric component of the hydrogel material, in resisting degradation (e.g., changes in shape, size, color, refractive index, ion permeability, equilibrium water content (EWC) or the like) that might otherwise be caused by exposure to electromagnetic radiation. The radiation resistant compound can resist degradation that could otherwise be caused by electromagnetic radiation anywhere in the electromagnetic spectrum. However, it is generally preferable that the radiation resistant compound resist degradation that would otherwise be caused by exposure to UV radiation (i.e., electromagnetic radiation having a wavelengths ranging from 100 nm or 150 nm and 400 nm), which can include near UV (i.e., wavelengths ranging from 300 nm to 400 nm), middle UV (i.e., wavelengths ranging from 200 nm to 300 nm), extreme UV (i.e., wavelengths ranging from 150 nm to 200 nm) or any combination thereof.

Advantageously, it has been found that particular UV chromophores provide UV protection to the hydrogel materials of the present invention or at least do not significantly increase the degradation of IOL materials due to UV exposure. In particular, benzotriazoles of the present invention have been shown to provide such characteristics.

Preferred benzotriazoles include, without limitation, substituted 2-hydroxyphenyl benzotriazole UV absorbers. UV chromophores suitable for use in the ophthalmic device materials of the present invention are represented by formula (A).

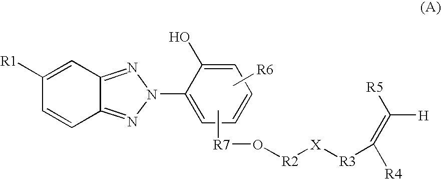

wherein for formula (A)
R₁ is a substituted or unsubstituted C₁-C₆ alkyl, a halogen, OH, C₁-C₁₂ alkyloxy, optionally substituted phenoxy, or optionally substituted napthyloxy, where the optional substituents are C₁-C₆ alkyl, C₁-C₆ alkoxy, OH, —(CH₂CH₂O)ₙ—, or —(CH₂CH(CH₃)O)ₙ—;
R₂ is a C₁-C₁₂ alkyl, (CH₂CH₂O)ₙ, (CH₂CH(CH₃)O)ₙ, or CH₂CH₂CH₂(Si(CH₃)₂O)ₘSi(CH₃)₂CH₂CH₂CH₂;
X is nothing if R₂ is (CⱼH₂CH₂O)ₙ or (CH₂CH(CH₃)O)ₙ, otherwise X is O, NR₄, or S;
R₃ is nothing, C(═O), C(═O)CⱼH₂ⱼ, C₁-C₆ alkyl, phenyl, or C₁-C₆ alkylphenyl;
R₄ is H or methyl;
R₅ is H, C₁-C₆ alkyl, or phenyl;
R₆ is H, C₁-C₁₂ alkyl, or C₁-C₁₂ alkyloxy (e.g., methoxy);
R₇ is C₁-C₆ alkyl or nothing;
m is 1-9;
n is 2-10; and
j is 1-6.

More particularly preferred UV chromophores, which are also of formula (A), suitable for use in the ophthalmic device materials of the present invention are represented by formula (I).

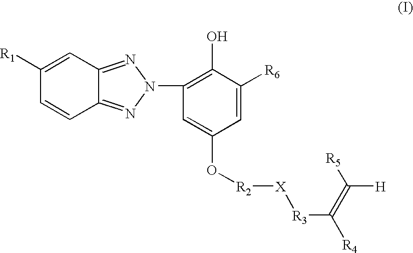

wherein for formula (I)
R₁ is a halogen, OH, C₁-C₁₂ alkyloxy, optionally substituted phenoxy, or optionally substituted napthyloxy, where the optional substituents are C₁-C₆ alkyl, C₁-C₆ alkoxy, OH, —(CH₂CH₂O)ₙ—, or —(CH₂CH(CH₃)O)ₙ—;
R₂ is a C₁-C₁₂ alkyl, (CH₂CH₂O)ₙ, (CH₂CH(CH₃)O)ₙ, or CH₂CH₂CH₂(Si(CH₃)₂O)ₘSi(CH₃)₂Cl₄₂CH₂CH₂;
X is nothing if R₂ is (CH₂CH₂O)ₙ or (CH₂CH(CH₃)O)ₙ, otherwise X is O, NR₄, or S;
R₃ is nothing, C(═O), C(═O)CⱼH₂ⱼ, C₁-C₆ alkyl, phenyl, or C₁-C₆ alkylphenyl;
R₄ is H or methyl;
R₅ is H, C₁-C₆ alkyl, or phenyl;
R₆ is H or C₁-C₁₂alkyl;
m is 1-9;
n is 2-10; and
j is 1-6.
Preferably in formula (I) and/or (A),
R₁ is Cl, Br, C₁-C₄ alkoxy, or phenoxy;
R₂ is C₁-C₆ alkyl;

### X is O or NR₄;

R₃ is C(═O) or C₁-C₆ alkylphenyl;
R₄ is H or methyl;

### R₅ is H; and

R₆ is C₄-C₁₂ t-alkyl.
Most preferably in formula (I) or (A),
R₁ is methoxy;
R₂ is C₂-C₃ alkyl;

### X is O;

### R₃ is C(═O);

R₄ is H or methyl;

### R₅ is H; and

R₆ is t-butyl.

The compounds of formula (A) and (I) can be made using methods known in the art. Two preferred compounds of formulas (A) and (I) are 2-{2′-Hydroxy-3′-tert-butyl-5′[3″-(4′″-vinylbenzyloxy)propoxy]phenyl}-5-methoxy-2H-benzotriazole:

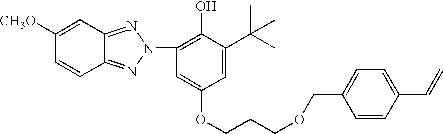

and 2-[2′-hydroxy-3′-tert-butyl-5′-(3″-methacryloyloxypropoxy)phenyl]-5-methoxy-2H-benzotriazole:

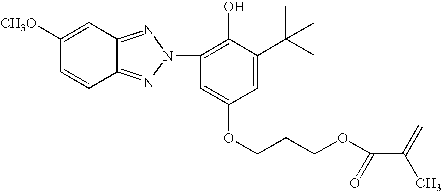

In preferred embodiments, the UV chromophores of the present invention provide a transmission cut-off above a wavelength of 385 and typically provide cut-off in the short wavelength visible (410-430 nm) region of the electromagnetic spectrum. These chromophores can then provide desired protection to the human tissued and/or the IOL material from UV radiation (<400 nm). The benzotriazoles above are examples of such UV chromophores. As such, these UV chromophores can also be referred to as UV/short wavelength visible light absorbers.

The device materials of the present invention can also comprise a polymerizable yellow dye that attenuates medium- to long-wavelength (430-500 nm) blue light. Such dyes and useful UV chromophores are described in commonly owned U.S. patent application Ser. No. 11/871,411, titled Intraocular Lenses with Unique Blue-Violet Cutoff and Blue Light Transmission Characteristics, filed Oct. 12, 2007, which is fully incorporated herein for all purposes.

Unless otherwise specified, “cut-off” means the wavelength at which light transmission does not exceed 1%. “1% cut-off” means the wavelength at which light transmission does not exceed 1%. “10% cut-off” means the wavelength at which light transmission does not exceed 10%.

As an additional advantage, it has been found that these benzotriazoles can be effective for resisting degradation due to radiation even when used in relatively low concentrations. Thus, it is contemplated that an effective amount of benzotriazole in the hydrogel material is less than 3% by weight, more typically less than 1% by weight and even possibly less than 0.5% by weight of the hydrogel material. The amount of benzotriazole is typically greater than about 0.02% by weight and even more typically greater than about 0.1% by weight of the hydrogel material. It should be understood, however, that these weight percentages for the radiation resistant compound do not limit the amount of radiation resistant compound that can be used within the scope of the present invention, unless otherwise specifically stated.

Advantageously, use of benzotriazoles of formula (I), particularly when used at lower concentrations, can provide the hydrogel material or IOL with enhanced ion permeability, enhanced EWC, enhanced extractables. In preferred embodiments, the hydrogel material of the present invention has an Ion Diffusion Coefficient (IDC) that is at least 15×10⁻⁷, more particularly at least 17×10⁻⁷ or at least 18×10⁻⁷, and even possibly at least 20×10⁻⁷ cm²/sec at 35° C. As is understood, the Ion Diffusion Coefficient is indicative of ion permeability. The coefficients given are for the diffusion of chloride ions using sodium chloride solutions. Methodology for determining Ion Diffusion Coefficient is provided below.

Additionally or alternatively, the hydrogel material can have an EWC percentage that is at least 50% more typically at least 53% and even possibly at least 55%. It is also contemplated that the hydrogel material can have an extractables percentage that is at least 13%. EWC percentage and extractables percentage are determined in accordance with gravimetric methods. It is to be noted that these values are pre-irradiation values, however, these values will also be enhanced in the post-irradiation particularly where the IOL material resists degradation from UV radiation.

EWC percentage can be determined for the present invention according to the following protocol: 1) weighing the hydrogel material in a fully or substantially fully (i.e., less than 1% by weight water) dehydrated state to get the dehydrated weight (W_{d}); 2) submerging the hydrogel material in purified de-ionized water (e.g., in a vial) for at least 24 hours at 37° C. to fully hydrate the material; and 3) weighing the fully hydrated material to get the fully hydrated weight (Wₕ). Then, the following equation is used to determine EWC percentage:

EWC percentage=((Wₕ−W_{d})/Wₕ)×100

It is to be understood that this type of UV protection is particularly desirable for PC Phakic IOLs. In particular, PC Phakic IOLs are typically located within the eye for extended periods of time (e.g., greater than 6 months, a year, several years or more) as opposed to, for example, disposable contact lenses. As such, it is highly desirable for these types of lenses to exhibit longer term resistance to degradation caused by radiation exposure. Further, it is particularly desirable to provide this protection to hydrogel PC Phakic IOLs since PC Phakic IOLs are typically disposed in the posterior chamber of the eye adjacent the natural crystalline lens of the eye and hydrogel materials have proven to be one of the few materials suitable for application in this location. Such PC Phakic IOLs will typically include, although not necessarily required, haptics that are angled to assist in fixing the IOL in the PC chamber.

Moreover, because of the properties discussed above, particularly the ion permeability, circulation of natural aqueous material to the eye can be enhanced. This is particularly important for PC Phakic IOLs and may even allow an IOL of the present invention with the posterior chamber to temporarily or more permanently contact or reside upon the natural crystalline lens of the eye rather than being located away from such natural crystalline lens.

It is additionally contemplated that the IOLs of the present invention can include a variety of additional or alternative ingredients, features or otherwise. Examples include, without limitation, coating materials, pharmaceuticals (therapeutic agents), cell receptor functional groups, protein groups, viscosity agents (e.g., thickeners or thinners), diluents, combinations thereof or the like.

IOLs of the present invention can be formed using multiple different techniques or protocols. According to one preferred protocol, the monomers (e.g., comonomers) of the present invention, the curing agent and optionally curing agent accelerator, the radiation resistant compound and any other desired ingredients are combined together to form a master batch. The master batch is then exposed to a stimulus (e.g., an ambient condition such as heat or light (e.g., blue light)) which initiates polymerization and cross-linking of the monomers. The initiated masterbatches can be cast into wafers of desired geometry and can be secured in cure fixtures for forming the IOLs.

The cast wafers are then typically cured through extended exposure to an ambient condition such as heat, light (e.g., blue light) or both. For example, in one embodiment, the cast wafers are exposed to an elevated temperature (e.g., about 70° C.) for a first period of time (e.g., about 2 hours) and then ramped up to a second temperature (e.g., about 110° C.) for a second period of time (e.g., at least 10 minutes). In a second exemplary embodiment, the wafers are cured using blue light at a wavelength of about 405 nm to about 415 nm for a first period of time (e.g., about 3 hours) and then exposed to an elevated temperature (e.g., about 110° C.) for a second period of time (e.g., about one hour). Preferably, the initiation, the curing or both are carried out in a low moisture (e.g., less than 1 ppm water), low oxygen (less than 100 ppm) environment.

Hydrogel materials formed in accordance with the present invention typically exhibit relatively high refractive indexes. The refractive index for a hydrogel material of the present invention at 25° C. is typically greater than about 1.410, more typically greater than about 1.415, still more typically greater than about 1.420 and even possibly greater than 1.44 or even 1.47 when the refractive index of the material (fully hydrated) is measured in accordance with BS EN ISO 11979-5:2000.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

### COMPARATIVE EXAMPLES

Table 1 below recites several formulations used to form hydrogels that were tested to determine photostability or resistance to degradation for exposure to UV radiation:

TABLE 1

| | | | | | NMP | | | | | Lucerin |
|---|---|---|---|---|---|---|---|---|---|---|
| | NVP | HEMA | PEMA | AMA | (diluent) | BHMA | UV13 | bnzfne | T21s | TPO |
| A (control) | 35.00% | 64.50% | — | 0.50% | 10.00% | — | — | — | 0.50% | — |
| B | 35.00% | 59.00% | — | 0.50% | 10.00% | 5.50% | — | — | 0.50% | — |
| C | 35.00% | 59.50% | — | 0.50% | 10.00% | 5.00% | — | — | 0.50% | — |
| D | 35.00% | 60.00% | — | 0.50% | 10.00% | 4.50% | — | — | 0.50% | — |
| E | 35.00% | 64.10% | — | 0.50% | 10.00% | — | 0.40% | — | 0.50% | — |
| F | 35.00% | 64.30% | — | 0.50% | 10.00% | — | 0.20% | — | 0.50% | — |
| G | 35.00% | 64.40% | — | 0.50% | 10.00% | — | 0.10% | — | 0.50% | — |
| H | 35.00% | 59.00% | — | 0.50% | 10.00% | — | — | 5.50% | 0.50% | — |
| I | 35.00% | 59.50% | — | 0.50% | 10.00% | — | — | 5.00% | 0.50% | — |
| J | 35.00% | 60.00% | — | 0.50% | 10.00% | — | — | 4.50% | 0.50% | — |
| K | 69.88% | — | 24.51% | 0.62% | — | 5.00% | — | — | 2.59% | — |
| L | 69.70% | — | 29.30% | 1.00% | — | — | — | — | 1.00% | 1.00% |
| M | 69.65% | — | 28.85% | 1.00% | — | — | 0.50% | — | 1.00% | 1.00% |

NVP—N-vinyl pirrolidone
HEMA—2-hydroxyethyl methacrylate
PEMA—poly(ethyl methacrylate)
AMA—allyl methacrylate
NMP—N-methyl-2-pyrrolidone
BHMA—2-[3-(2H-benzotriazol-2-yl)-4-hydroxyphenyl]ethyl methacrylate
UV13—2-(2′-hydroxy-3′-tert-butyl-5′-(3″-methacryloyloxy)propoxyphenyl]-5-methoxy-2H-benzotriazole
bnzfne—4-(2-acryloxyethoxy)-2-hydroxybenzophenone
T21s—tert-butyl peroxy-2-ethylhexanoate
Lucerin TPO—2,4,6-trimethylbenzoyldiphenylphosphine oxide

The formulations include a benzotriazol hydroxyphenyl ethyl methacrylate (BHMA), a substituted 2-hydroxyphenyl benzotriazole (UV-13) according to Formula I above or a benzonphenone (bnzfe) as a UV chromophore. With reference to table 2 below, it can be seen that the samples having UV-13 provide greater extractables percentage, greater EWC and greater ion permeability.

TABLE 2
Extractables, EWC, and ion permeability of PC Phakic UV materials

| | | | | | Ion Diffusion | |
|---|---|---|---|---|---|---|
| | | | | | Coefficient | |
| ID (UV absorber % | | | EWC | | D ((cm2/sec) × | |
| (w/w)) | ext. (%) | SD | (%) | SD | 10−7) | SD |
| Control (A) | 13.22 | 0.11 | 55.73 | 0.09 | 19.2 | 1.6 |
| BHMA5.5 (B) | 12.10 | 0.16 | 47.84 | 0.61 | 9.8 | — |
| BHMA5.0 (C) | 12.04 | 0.17 | 48.38 | 0.11 | 10.2 | — |
| BHMA4.5 (D) | 12.19 | 0.39 | 48.83 | 0.07 | 10.6 | — |
| UV130.4 (E) | 13.09 | 0.67 | 55.69 | 0.08 | 18.4 | 1.7 |
| UV130.2 (F) | 13.30 | 0.15 | 55.93 | 0.06 | 21.7 | 2.6 |
| UV130.1 (G) | 13.38 | 0.27 | 55.86 | 0.22 | 18.6 | 0.3 |
| BNZFNE5.5 (H) | 12.74 | 0.10 | 48.58 | 0.07 | 12.1 | — |
| BNZFNE5.0 (I) | 12.64 | 0.20 | 49.28 | 0.07 | 15.3 | — |
| BNZFNE4.5 (J) | 12.63 | 0.21 | 49.91 | 0.02 | 17.4 | — |

For comparative purposes, UV radiation testing was applied to control samples A and L as well as samples K and M. Testing was done according to ISO 11979-5:2006 standard for Ophthalmic Implants/Intraocular Lenses. After testing, sample K showed significant degradation by virtue of yellowing and marked difference in UV/Vis spectra after testing at ˜46 days of exposure ˜100 W/m2 UV-A at ˜37° C. as figured in accordance with the ISO Standard. In contrast, samples that included UV-13 or no UV chromophore at all did not exhibit similar degradation.

Further, Table 3 below shows diopter power measurements for sample A (i.e., a sample without UV Chromophore) and a sample substantially identical to sample E (i.e., a sample like Sample A, but including UV 13) prior to UV exposure, after a 10 yr equivalent UV exposure and after a 20 year equivalent exposure.

TABLE 3
Power Measurements of PC Phakic materials post-UV irradiation

| | | | | 10 yr, N = 3 lenses | | | | | |
|---|---|---|---|---|---|---|---|---|---|
| | N = 3 lenses ctrl | | | each | | | 20 yr, N = 6 lenses | | |
| | Initial | ctrl | Diff. | Initial | 10 yr | Diff. | Initial | 20 yr | Diff. |
| E UV13 0.5% | −9.84 | −9.95 | 0.11 | −9.96 | −9.85 | −0.11 | −9.78 | −9.71 | −0.07 |
| SD | 0.04 | 0.06 | 0.09 | 0.16 | 0.14 | 0.05 | 0.24 | 0.34 | 0.14 |
| A | −10.51 | −10.50 | −0.01 | −10.41 | −9.93 | −0.48 | −10.31 | −9.80 | −0.52 |
| SD | 0.13 | 0.46 | 0.47 | 0.13 | 0.33 | 0.22 | 0.23 | 0.27 | 0.24 |

As can be seen, the power measurements for sample E are substantially unchanged while the power measurements for sample A are significantly altered. As such, it appears that the UV 13 acts to protect the IOL material from degradation due to UV exposure.

Ion Diffusion Coefficient Measurements

Ion diffusion coefficients for hydrogel materials of the present invention can be determined using a solution separation system. In particular, a sample of the hydrogel material is disposed between a first solution having a relatively high concentration of sodium chloride (NaCl) and second solution having relatively low concentration of NaCl or no NaCl. Thereafter, one or more conductivity meters and conductivity probes are use to measure the change in conductivity of the first solution, the second solution or both. During such measurements, the first and second solutions should be continuously stirred and maintained at a temperature of 35° C. The ion diffusion coefficient (D) of the sample can then be determined by relating the conductivity of the second solution to the ion diffusion coefficient using Fick's Law and a mass balance. In particular, Fick's Law states that the flux per unit area (J) is proportional to the concentration (C) gradient measured normal to the cross section (x), i.e.

J=−D(φC/φx)

The conservation of mass balance mathematically states that the increase in concentration in one solution of the sample with respect to time (t) must correspond to an equal decrease in concentration of the other solution, taking into account the volumes (V) associated with each of the first and second solutions, i.e.

Vₕ(dCₕ/dt)+V(dC₁/dt)=0

where subscript h is the high concentration solution and subscript 1 is the low concentration solution. Using these principles and methodologies as well as good scientific calibration and washing, the skilled artisan will be able to determine the ion diffusion coefficient to a high degree of accuracy.

We claim:

1. An ophthalmic device, comprising;
an intraocular lens configured for insertion into an eye, wherein:
i. the lens is formed of a hydrogel material that is formed with cross-linked polymer that includes a cyclic nitrogen containing monomer; and
ii. the lens includes UV chromophore and the UV chromophore includes a benzotriazole according to the following formula: 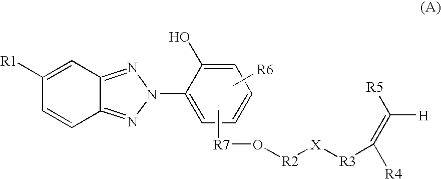
wherein for formula (A)
R₁ is a substituted or unsubstituted C₁-C₆ alkyl, a halogen, OH, C₁-C₁₂ alkyloxy, optionally substituted phenoxy, or optionally substituted napthyloxy, where the optional substituents are C₁-C₆ alkyl, C₁-C₆ alkoxy, OH, —(CH₂CH₂O)ₙ—, or —(CH₂CH(CH₃)O)ₙ—;
R₂ is a C₁-C₁₂ alkyl, (CH₂CH₂O)ₙ, (CH₂CH(CH₃)O)ₙ, or CH₂CH₂CH₂(Si(CH₃)₂O)ₘSi(CH₃)₂CH₂CH₂CH₂;
X is nothing if R₂ is (CH₂CH₂O)ₙ or (CH₂CH(CH₃)O)ₙ, otherwise X is O, NR₄, or S;
R₃ is nothing, C(═O), C(═O)CⱼH₂ⱼ, C₁-C₆ alkyl, phenyl, or C₁-C₆ alkylphenyl;
R₄ is H or methyl;
R₅ is H, C₁-C₆ alkyl, or phenyl;
R₆ is H, C₁-C₁₂ alkyl, or C₁-C₁₂ alkyloxy (e.g., methoxy);
R₇ is C₁-C₆ alkyl or nothing;
m is 1-9;
n is 2-10; and
j is 1-6.

2. An ophthalmic device as in claim 1 wherein the benzotriazole is of the following formula: 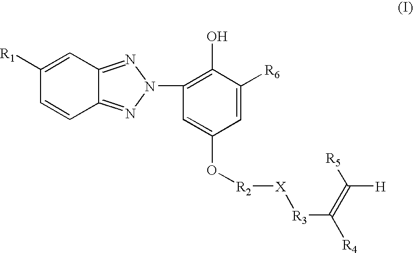
wherein:
R₁ is a halogen, OH, C₁-C₁₂ alkyloxy, optionally substituted phenoxy, or optionally substituted napthyloxy, where the optional substituents are C₁-C₆ alkyl, C₁-C₆ alkoxy, OH, —(CH₂CH₂O)ₙ—, or —(CH₂CH(CH₃)O)ₙ—;
R₂ is a C₁-C₁₂ alkyl, (CH₂CH₂O)ₙ, (CH₂CH(CH₃)O)ₙ, or CH₂CH₂CH₂(Si(CH₃)₂O)ₘSi(CH₃)₂CH₂CH₂CH₂;
X is nothing if R₂ is (CH₂CH₂O)ₙ, or (CH₂CH(CH₃)O)ₙ, otherwise X is O, NR₄, or S;
R₃ is nothing, C(═O), C(═O)CⱼH₂ⱼ, C₁-C₆ alkyl, phenyl, or C₁-C₆ alkylphenyl;
R₄ is H or methyl;
R₅ is H, C₁-C₆ alkyl, or phenyl;
R₆ is H or C₁-C₁₂ alkyl;
m is 1-9;
n is 2-10; and
j is 1-6.

3. An ophthalmic device as in claim 1 wherein:
R₁ is Cl, Br, C₁-C₄ alkoxy, or phenoxy;
R₂ is C₁-C₆ alkyl;
X is O or NR₄;
R₃ is C(═O) or C₁-C₆ alkylphenyl;
R₄ is H or methyl;
R₅ is H; and
R₆ is C₄-C₁₂ t-alkyl.

4. An ophthalmic device as in claim 1 wherein:
R₁ is methoxy;
R₂ is C₂-C₃ alkyl;
X is O;
R₃ is C(═O);
R₄ is H or methyl;
R₅ is H; and
R₆ is t-butyl.

5. An ophthalmic device as in claim 1 wherein the UV chromophore is 2-{2′-Hydroxy-3′-tert-butyl-5′[3″-(4′″-vinylbenzyloxy)propoxy]phenyl}-5-methoxy-2H-benzotriazole: 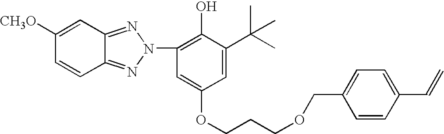
or 2-[2′-hydroxy-3′-tert-butyl-5′-(3″-methacryloyloxypropoxy)phenyl]-5-methoxy-2H-benzotriazole:

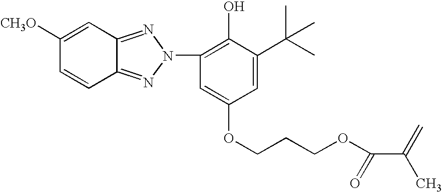

6. An ophthalmic device as in claim 1 wherein the UV chromophore significantly enhances resistance of the lens to degradation by electromagnetic radiation.

7. An ophthalmic device as in claim 1 wherein the cross-linked polymer includes vinyl methacrylate.

8. An ophthalmic device as in claim 7 wherein the vinyl methacrylate is an NVP methacrylate copolymer.

9. An ophthalmic device as in claim 8 wherein the vinyl methacrylate includes NVP-co-hydroxylmethacrylate, NVP-co-aryl mecthacrylate or a combination thereof.

10. An ophthalmic device as in claim 1 wherein the hydrogel material has a refractive index that is at least 1.4.

11. An ophthalmic device as in claim 1 wherein the hydrogel material includes at least 0.02% by weight but less than 1% by weight UV chromophore.

12. An ophthalmic device as in claim 1 wherein the hydrogel material includes less than 0.5% by weight UV Chromophore.

13. An ophthalmic device as in claim 1 wherein the eye has an anterior chamber and a posterior chamber and the lens is sized and shaped for insertion into the posterior chamber of the eye as a PC Phakic lens.

14. An ophthalmic device as in claim 1 wherein the ion diffusion coefficient of the hydrogel material is at least 17×10⁻⁷ cm²/sec at 35° C.

15. An ophthalmic device as in claim 1 wherein the equilibrium water content of the hydrogel material is at least 50%.

16. An ophthalmic device, comprising;
an intraocular lens configured for insertion into an eye, the eye having an anterior chamber and a posterior chamber and the lens being sized and shaped for insertion into the posterior chamber of the eye as a PC Phakic lens wherein:
i. the lens is formed of a hydrogel material that is formed with cross-linked polymer that includes a cyclic nitrogen containing monomer that is NVP-co-hydroxylmethacrylate, NVP-co-aryl mecthacrylate or a combination thereof; and
ii. the lens includes UV chromophore and the UV chromophore includes a benzotriazole according to the following formula: 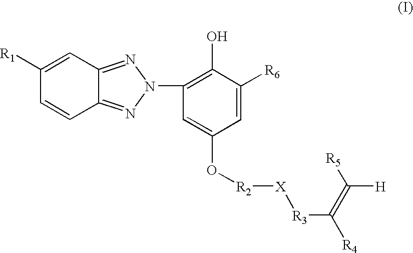
wherein:
R₁ is a halogen, OH, C₁-C₁₂ alkyloxy, optionally substituted phenoxy, or optionally substituted napthyloxy, where the optional substituents are C₁-C₆ alkyl, C₁-C₆ alkoxy, OH, —(CH₂CH₂O)ₙ—, or —(CH₂CH(CH₃)O)ₙ—;
R₂ is a C₁-C₁₂ alkyl, (CH₂CH₂O)ₙ, (CH₂CH(CH₃)O)ₙ, or CH₂CH₂CH₂(Si(CH₃)₂O)ₘSi(CH₃)₂CH₂CH₂CH₂;
X is nothing if R₂ is (CH₂CH₂O)ₙ or (CH₂CH(CH₃)O)ₙ, otherwise X is O, is NR₄, or S;
R₃ is nothing, C(═O), C(═O)CⱼH₂ⱼ, C₁-C₆ alkyl, phenyl, or C₁-C₆ alkylphenyl;
R₄ is H or methyl;
R₅ is H, C₁-C₆ alkyl, or phenyl;
R₆ is H or C₁-C₁₂alkyl;
m is 1-9;
n is 2-10; and
j is 1-6.

17. An ophthalmic device as in claim 16 wherein:
R₁ is Cl, Br, C₁-C₄ alkoxy, or phenoxy;
R₂ is C₁-C₆ alkyl;
X is O or NR₄;
R₃ is C(═O) or C₁-C₆ alkylphenyl;
R₄ is H or methyl;
R₅ is H; and
R₆ is C₄-C₁₂ t-alkyl.

18. An ophthalmic device as in claim 16 wherein the UV chromophore is 2-{2′-Hydroxy-3′-tert-butyl-5′[3″-(4′″-vinylbenzyloxy)propoxy]phenyl}-5-methoxy-2H-benzotriazole: 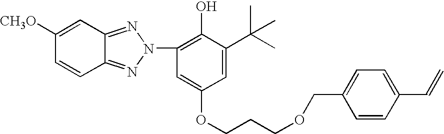
or 2-[2′-hydroxy-3′-tert-butyl-5′-(3″-methacryloyloxypropoxy)phenyl]-5-methoxy-2H-benzotriazole:

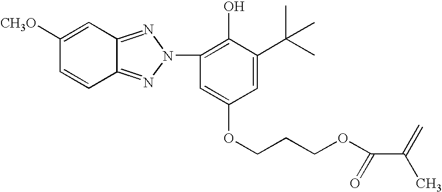

19. An ophthalmic device as in claim 16 wherein the hydrogel material includes less than 0.5% by weight UV Chromophore.

20. An ophthalmic device as in claim 16 wherein the ion diffusion coefficient of the hydrogel material is at least 17×10⁻⁷ cm²/sec at 35° C. and wherein the equilibrium water content of the hydrogel material is at least 50%.
